(19) United States
(12) Patent Application Publication
(10) Pub. No.: US 2002/0197637 A1
(43) Pub. Date: Dec. 26, 2002

(54) Process and compositions for protection of nucleic acids

(76) Inventors: Richard C. Willson III, Houston, TX (US); Jason C. Murphy, North Wales, PA (US)

Correspondence Address: RICHARD COALE WILLSON JR, 3205 HARVEST MOON DR, STE 200, PALM HARBOR, FL, 34683-2127, US

(21) Appl. No.: 10/158,753

(22) Filed: May 30, 2002

Related U.S. Application Data
(60) Provisional application No. 60/295,350, filed on Jun. 2, 2001.

(51) Int. Cl. C12Q 1/68; C07H 21/04; C12N 1/08

(52) U.S. Cl. 435/6; 435/270; 536/25.4

(57) ABSTRACT

A mechanical cell lysis technique involving the use of compaction protection technology to shield nucleic acids during mechanical lysis. Mechanical lysis is an efficient and widely used method of liberating the contents of microbial cells, but the shear sensitivity of large nucleic acids impairs the application of this technique to DNA purification. The invention uses compaction agents, small polycations that condense nucleic acids, to protect DNA from shear damage and allow mechanical lysis to be used in chromosomal and plasmid DNA purification. In addition to protecting DNA during lysis, compaction allows DNA to be pelleted with the insoluble cell debris, washed, and resolubilized to yield an enriched DNA product. Highly shear-sensitive nucleic acid molecules such as large plasmids and BACs can also be protected during lysis. An added benefit is that lysate viscosity is greatly reduced, allowing for reduced volumes compared to alkaline lysis.

### BACKGROUND OF THE INVENTION

This application claims priority of provisional application 60/295,350 filed Jun. 1, 2001 (011APR) and of U.S. Ser. No. 09/841,763 filed 24 Apr. 2, 2001 (009MUS) and of U.S. Ser. No. 09/609,996 filed Jul. 3, 2000 (009AUS) which claims priority of provisional application 60/143,768 filed Jul. 12, 1999 (009APR).

1. Field of the Invention

The present invention related to the general field of biochemial separations, and to apparatus for their practice, generally classified in U.S. Patent Class 435.

Acknowledgments

This research was funded in part by grants to R. C. W. and G. E. F. from NASA through the National Space Biomedical Research Institute, the Environmental Protection Agency (R825354-01-0), and the Welch Foundation.

2. Description of the Prior Art

Prior art in the field includes a study of mechanical lysis of plasmid DNA that determined in best case scenarios about 90% of plasmid DNA can be recovered undamaged, but usually much less. In addition, prior art includes a large body of literature on the structure and function of compaction agents.

Including

1. Sambrook, J., and Russell, D. W. 2001. Molecular Cloning, a Laboratory Manual.

Third edition, Cold Spring Harbor Laboratory Press which summarizes the prior art in nucleic acid purification.

2. Holmes, D. S. and Quigley, M. 1981. A rapid boiling method for the preparation of bacteria plasmids. Anal. Biochem. 114:193-197 which describes a heat-based method of plasmid purification, not using protection agents.

3. Scopes R. K. 1994. Protein purification: principles and practice. New York: Springer Verlag. 380 p., which summarizes methods of purifying proteins, including mechanical lysis and other methods potentially applicable in the method of the present invention.

4.Carlson A, Signs M, Liermann L, Boor B, Jem K J. 1995. Mechanical Disruption of Escherichia coli for Plasmid Recovery Biotechnol. Bioeng, 48:303-315, which summarizes experience with plasmid isolation after mechanical lysis without protection.

5.Murphy J C, Wibennmeyer J A, Fox G E, Willson R C. 1999. Purification of plasmid DNA using selective precipitation by compaction agents. Nature Biotechnol. 17:822-823, which describes precipitation of plasmid DNA by compaction agents not used as protecting agents.

6.Murphy J C, Fox G E, Willson R C. 2001. RNA isolation and fractionation with compaction agents. Anal. Biochem. 295: 143-148, which describes precipitation of RNA by compaction agents not used as protecting agents.

7.Wilson R W, Bloomfield V A. 1979. Counterion-induced condensation of deoxyribonucleic acid. A light-scattering study. Biochemistry. 18:2192-2196, which describes conformational modification of DNA by compaction agents not used as protecting agents.

8.Hoopes B C, McClure W R 1981. Studies on the selectivity of DNA precipitation by spermine. Nucleic Acids Res. 9:.5493-5504which describes precipitation of DNA by compaction agents not used as protecting agents.

9.Gosule L C, Schellman J A. 1976. Compact form of DNA induced by spermidine. Nature. 259:333-335, which describes conformational modification of DNA by compaction agents not used as protecting agents.

3. Problems Presented by Prior Art

Currently, the types of lysis techniques described herein are not viable for use in nucleic acid processing due to the inherent shear sensitivity of high molecular weight nucleic acid molecules (e.g. plasmid DNA and genomic DNA). Processing sufficiently aggressive to give effective lysis of a high proportion of cells degrades a large fraction of the desired nucleic acid. More conservative treatments which spare a larger portion of the nucleic acid result in low efficiency of cell lysis. This has led to the widespread use of non-shear-based lysis methods, which often employ hazardous or expensive reagents, give poor yields, are non-robust, and/or involve processing at excessively large volumes.

Introduction

With a number of DNA vaccines and gene therapy products now in clinical trials, there is strong demand for improved large-scale DNA separation techniques. One major area of concern during large-scale DNA production is the initial host cell lysis step. Current lysis techniques for DNA production require caustic solutions, enzymes, and/or heat to liberate DNA from bacterial cells (1,2). In addition, all of these lysis methods require large volumes to keep viscosity within practical limits.

Mechanical lysis techniques used in protein recovery are generally more efficient than the methods customarily used in the liberation of DNA from cells (3). Large nucleic acids such as plasmid and chromosomal DNA, however, are sheared and fragmented by most mechanical lysis techniques (4). We have previously shown that condensation of DNA to a more compact form can enhance its adsorption (JM, GF, RW, in press), and can serve as the basis for selective precipitation and fractionation of DNA and RNA (5,6). Compaction employs small synthetic polycations, such as spermine and spermidine, to induce reversible conformational changes and/or precipitation of nucleic acids (7,8).

Here we show that compaction agent-induced condensation of DNA enhances yields after mechanical lysis and that the precipitated DNA can then be quickly separated in the presence of insoluble cell debris. In addition, the diminished viscosity of the compaction protected lysate allows lysis to take place in a reduced volume when compared to traditional alkaline lysis techniques. This benefit of operation in reduced volumes can also be applied to non-mechanical lysis techniques. Compaction protection can also increase yields of shear-sensitive nucleic acids in lysis methods not primarily based on shear.

Bioseparations, especially separation of RNA from DNA or vice versa, are conventionally accomplished in bench scale or larger pilot plants in which a fermentation is carried out to produce cell mass which is lysed, then exposed to filtration and nucleases are used to reduce unwanted nucleic acid populations (e.g. the use of ribonuclease (RNAse) in plasmid purification).

Generally, after these initial solution phase purification steps, the effluent products are further purified by chromatographic columns (e.g. anion exchange or size exclusion chromatogaphy), often with samples being analyzed and results subjected to quality control feedback techniques. Such procedures can take a day or more for a single run or batch on a single mixture, assuming the optimum conditions, concentrations, etc. The present invention permits the separation of dozens of feed mixtures in a single set-up, often in less time than required for a single separation by conventional methods.

Further, when practiced in its preferred embodiments, the invention can sharply reduce the production costs (costs per milligram of purified DNA product produced).

### SUMMARY OF THE INVENTION

Mechanical cell lysis techniques have been used in the production of proteins for 5 decades (Scopes, 1993). However, when bacteria are mechanically lysed, large nucleic acids such as plasmid and chromosomal DNA are mechanically sheared and fragmented. The inventors have developed a means of protecting nucleic acids from these shear effects, allowing mechanical lysis to be done on cells quickly and efficiently. In addition, because the protection of the large, double-stranded nucleic acids can also involve their selective precipitation, a quick purification is also possible.

### DETAILED DESCRIPTION OF THE INVENTION

The inventors use compaction agents to perform this protection method. They are cost effective, synthetically produced, small cationic molecules. The inventors preferably use spermidine to protect these nucleic acids. This compaction protection is accomplished by adding a compaction agent (in this case spermidine) to the lysis buffer in which the bacterial paste is resuspended for the mechanical lysis. This buffer can also include either a detergent or an organic solvent to loosen up the bacterial cell envelope so the compaction agent can pass into the cell prior to lysis.

Additional applications include general protection of the acids from shear and enzymatic degradation during lysis by sonication, heat, chemicals, etc. and during handling (sterile filling) and long-term storage. Each of these parameters is discussed below:

Cell Mass

The starting material is often a mass of cells prepared by fermentation or cell culture, isolated from the environment, or derived from tissues. The cells are then disrupted so the nucleic acids are freed, forming a lysate. The lysate then optionally undergoes an alkaline lysis, precipitation, adsorption or other process to form a purified product. A variety of cell types can be used as feed for this whole process, with bacterial, yeast, or other eukaryotic cells. Gram-negative and Gram-positive being preferred and Gram-negative being most preferred.

Product

The product of the invention can be purified DNA, most preferably plasmid DNA, e.g. as used in preparation of influenza or other vaccines.

In general, the selective precipitation of the invention can be applied to all bacteria (Gram-negative, Gram-positive and Archaea), all eukaryotes (such as yeast and human cells), recombinant cells, and all synthetic nucleic acids. The invention can separate BACs (bacteria artificial chromosomes) YACs (yeast artificial chromosomes). BACs and YACs are very large plasmids in bacteria and yeast, used in sequencing projects. The invention can also be applied to the production of cosmids (and very large plasmids in general), artificial chromosomes, chromosomal DNA, and phage and other viral DNA, and the detection of protein-nucleic acid binding and viruses.

Compaction Agents

The compaction agents are preferably small, nonchaotropic, cationic molecules, which bind in either the major or minor grooves of a double stranded RNA or DNA molecule, reducing the volume occupied by the nucleic acid. Some compaction agents function in vivo to package genomic DNA into sperm, and can serve a similar function in the delivery of DNA pharmaceuticals.

Compaction of DNA involves charge neutralization in combination with stabilization of inter-helix interactions. The compaction agent binds in either the major or minor groove, in proximity to the negatively charged phosphate groups.

In general, there will be added about 0.1 to 40, most preferably about 0.2 to 15 mM of a compaction agent, preferably selected from the group consisting of: basic polypeptides (e.g. polylysine), polyamines (e.g. protamine, spermidine, spermine, putrescine, cadaverine, etc.), trivalent and tetravalent metal ions (e.g. hexammine cobalt, chloropentamine cobalt, chromium (III)), netropsin, distamycin, lexitropans, DAPI (4′,6 diamino 2-phenylindol), berenil, pentamidine, manganese chloride. At present knowledge, the moieties in parenthesis will be more preferred, but any other molecule that can be used to compact DNA via the mechanism described above may be used according to the product to be produced and the cell mass available.

Many other agents may be considered compaction agents and these include: basic polypeptides (i.e. polylysine), polyamines (i.e. protamine, spermidine, spermine, cadaverine, etc.), trivalent and tetravalent metal ions (i.e. hexammine cobalt, chloropentamine cobalt, chromium (III)) netropsin, distamycin, lexitropans, DAPI (4′, 6 diamino 2-phenylindol), berenil, pentamidine, manganese chloride, or any other molecule that can be used to compact DNA via the mechanism described above (see references 1-7, 9, 17-19, 36, 37, 38). Also any protein having multiple binding domains for nucleic acids can potentially, for large complexes, result in the precipitation of nucleic acids.

For the protection of plasmid DNA, genomic DNA, and other large double-stranded nucleic acids, the most preferred compaction agent is spermidine.

Lysing Agent

Preferred methods of lyzing include: detergent, nonionic detergent, heat, French press, sonicator, homogenizer, microfluidizer, freeze/thaw, toluene, organic solvent, amines, quaternary amines, enzyme, lysozyme, lysostaphin, osmotic shock, chloroform, extruder, bead mill, microneedles, acid, alkali, phage protein. Of these the preferred lyzing agents include: French press, homogenizer, detergent, bead mill, microfluidizer, freeze/thaw, enzyme, and heat; and the most preferred include heat, detergent, homogenizer, and microfluidizer.

Analysis Methods and Results

FIG. 1 shows compaction protection of nucleic acids during French press lysis at an average pressure of 11,000 psi. Lane 1 is the control unprotected French pressed lysate (in a buffer containing 0.5% Brij 58 in 20 mM Tris HCl at pH 8.0), and Lane 2 is the spermidine protected lysate (0.5% Brij 58 with 30 mM spermidine in 20 mM Tris HCl at pH 8.0). For analysis of these crude lysates, each sample was precipitated with 0.7 volumes of isopropanol and resuspended in 1× TAE. Very little DNA remains intact in the control but the plasmid and genomic DNA are visible after the compaction protected French press lysis. The compaction protection solution also reduces fragmentation of DNA (both genomic and plasmid). This is an advantage because these randomly-sheared fragments can be difficult to completely separate from intact plasmid DNA.

Because compaction protection involves DNA condensation agents, the method can be integrated with our previous work on compaction precipitation (5, 6, under Prior Art, above) into an efficient lysis/purification protocol by carrying out the RNA/DNA separation in the presence of insoluble cell debris from the lysis.

FIG. 2 shows integrated compaction protection lysis/purification of plasmid DNA. Lane 1 is the spermidine protected lysate (0.5% Brij 58with 15 mM spermidine in 20 mM Tris HCl at pH 8.0) after lysis by French press at an average pressure of 4000 psi; Lane 2 is the RNA-rich supernatant from centrifugation of the lysate in Lane 1 (for analysis the samples in Lanes 1 and 2 were precipitated with 0.7 volumes of isopropanol and resuspended in 1× TAE); Lane 3 is the centrifuged Lane 1 lysate after compaction agent removal by washing with 50% ethanol with 600 mM NaCl and 10 mM EDTA, followed by a 70% EtOH wash and resuspension in 1× TAE. This material is enriched in plasmid and genomic DNA and depleted of RNA and other contaminants. The DNA remains precipitated until the purified material is resuspended. Viscosities remain low throughout the process, allowing the lysis and subsequent purification to take place in a greatly reduced volume compared with traditional lysis methods. Yields in these un-optimized processes are not yet accurately known, though they are evidently substantial (similar to that of alkaline lysis), and much greater than by mechanical lysis without compaction protection. Though much less pure than the typical alkaline lysis product, the product can readily be further processed to obtain pure plasmid DNA, though the overall process efficiency compared to alkaline lysis remains to be established.

Besides the French press, we have also evaluated the application of compaction protection to other mechanical cell lysis techniques. Glass beads have been used in a vortexer mini-prep application to directly release plasmid and gene DNA from both fresh and frozen E. coli. In addition, sonication of bacterial cells in the compaction protection solution has been found to effectively release DNA (results not shown).

Compaction agent-induced protection can allow liberation of nucleic acids from cells using existing equipment for protein purification. This lysis not only simplifies the purification of large nucleic acids but also avoids the high viscosities possible when nucleic acids are released into a small volume of liquid. The resulting low-viscosity lysates are advantageous starting point for purification of other cell-derived products, especially proteins. Potential further applications include protection of nucleic acids during sterile filling, for storage, and in protection of nucleic acids during heat-based lysis. Compaction protection may also find applications in the purification of yeast artificial chromosomes (YACs), bacterial artificial chromosomes (BACs), and other sensitive acid molecules.

### DESCRIPTION OF DRAWINGS

FIG. 1 shows compaction protection of nucleic acids during French press lysis at an average pressure of 11,000 psi. Lane 1 is the control unprotected French pressed-lysate (in a buffer containing 0.5% Brij 58 in 20 mM Tris HCl at pH 8.0), and Lane 2 is the spermidine-protected lysate (0.5% Brij 58 with 30 mM spermidine in 20 mM Tris HCl at pH 8.0). For analysis of these crude lysates, each sample was precipitated with 0.7 volumes of isopropanol and resuspended in 1× TAE.

FIG. 2 shows integrated compaction protection lysis/purification of plasmid DNA. Lane 1 is the spermidine protected lysate (0.5% Brij 58 with 15 mM spermidine in 20 mM Tris HCl at pH 8.0) after lysis by French press at an average pressure of 4000 psi; Lane 2 is the RNA-rich supernatant from centrifugation of the lysate in Lane 1 (for analysis the samples in Lanes 1 and 2 were precipitated with 0.7 volumes of isopropanol and resuspended in 1× TAE); Lane 3 is the centrifuged Lane 1 lysate after compaction agent removal by washing with 50% ethanol with 600 mM NaCl and 10 mM EDTA, followed by a 70% EtOH wash and resuspension in 1× TAE.

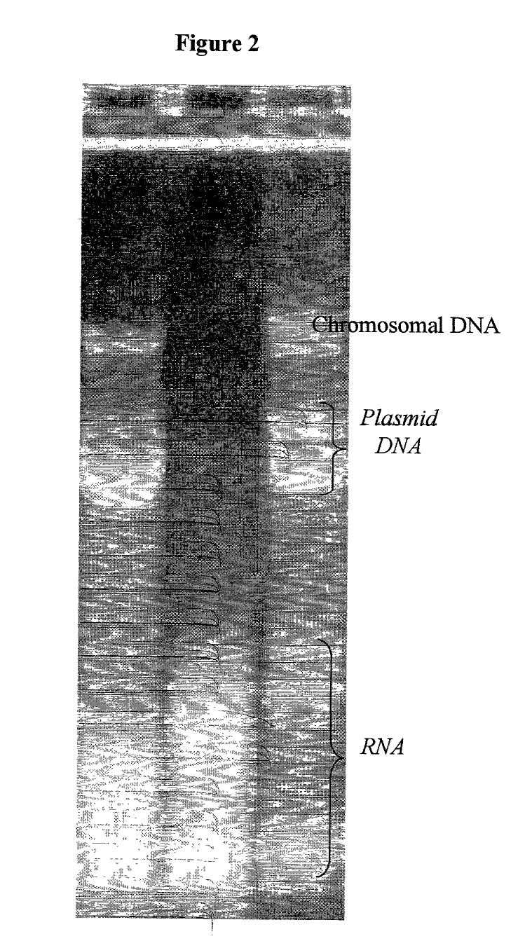

Table A lists Preferred, More Preferred, and Most Preferred embodiments of a number of parameters of the invention.

### EXAMPLES

### Example 1

### Mechanical Lysis of Bacterial Cells for the Recovery of Plasmid DNA

E. coli JM109 strain containing pCMV Sport, B gal plasmid DNA (Gibco BRL) grown in Pseudomonas Media 187 (per liter of media add 10 g tryptone, 10 g yeast extract, 15 g K2HPO4, 10 g glycerol, 5 mL salts solution to 1 L of distilled water where the salts solution contains 4.0 g MgSO4*7H2O, 0.2 g NaCl, 0.4 g FeSO4*7H2O, and 0.2 g MnSO4*4H2O in 100 mL of H2O) at 37° C. in a 20 L Applikon fermentor (20 liter in situ sterilizable bioreactor model number Z611120001). Overall fermentation time continues for about 12 hours and the cells grow to an OD600 of about 20. The fermentor is harvested and the cells are pelleted at 4000 rpm in a Beckman centrifuge (6 L capacity rotor) for 30 minutes. Then the resulting pellets are optionally placed into plastic bags and heat-sealed to make crisps. The yield of the fermentation is approximately 450 grams of wet cell paste. This bacterial paste is then taken and resuspended in an aqueous compaction protection solution, 10 mL per gram of wet cells, composed of a nonionic detergent (1% Brij 58), spermidine trihydrochloride (optimally 10 mM), and 30 mM Tris HCl at pH 8.0.

This solution is vortexed to suspend the bacteria in the buffer and then run through a French cell press (SLM Aminco, cat # PC-160) at an average pressure of 11,000 psi. These lysed cells were then taken and immediately centrifuged at 10,000 × g in a Beckman centrifuge for 15 minutes. Next, the supernatant is decanted and a solution (50 % EtOH, 300 mM NaCl, 20 mM EDTA) is contacted with the pellet to strip the compaction agents from the DNA now pelleted with the cellular waste. The bottle is vortexed, centrifuged, and the stripping solution is decanted. The pellet is then washed with 70% EtOH, decanted and then resuspended (the now soluble nucleic acids) in 1× TAE (40 mM Tris HCl, 1 mM EDTA, 40 mM NaAc at pH 8.0). The sample is then vortexed, centrifuged, and the supernatant is decanted to a new tube.

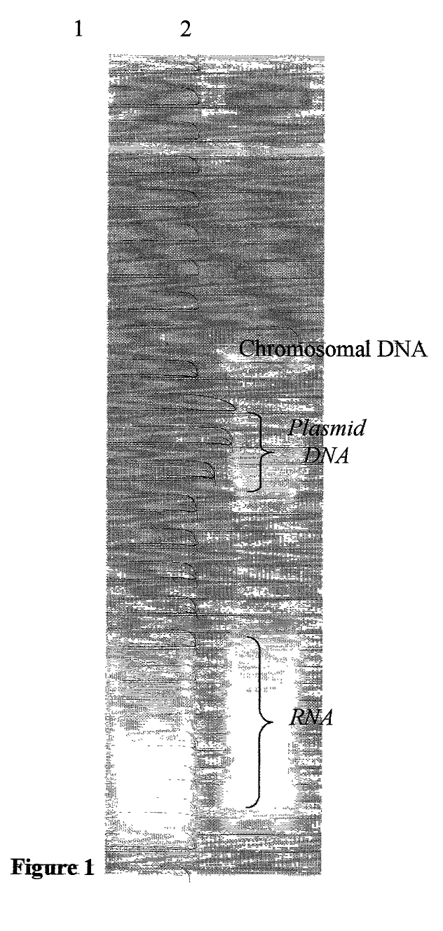

Gel electrophoresis and spectrophotometeric measurements can be run on the samples to determine purity and the integrity of the nucleic acids (mainly plasmid DNA) after lysis. The control experiment pictured in FIG. 1, Lane 1, showed no intact plasmid or chromosomal DNA while Lane 2 shows the compaction-protected plasmid DNA and chromosomal DNA highly intact after this separation.

### Example 2

### Mechanical Lysis of Bacterial Cells for the Recovery of genomic DNA

Using the same procedure detailed in Example 1 a lysate can be used to purify genomic DNA from bacterial cells. Bacterial cell paste is then taken and resuspended in an aqueous compaction protection solution, 10 mL per gram of wet cells, composed of a non-ionic detergent (1% Brij 58), spermidine trihydrochloride (optimally 10 mM, and 20 mM Tris HCl at pH 8.0.

This solution is vortexed to resuspend the bacteria in the buffer and then run through a French cell press (SLM Aminco, cat # PC-160) at an average pressure of 11,000 psi. These lysed cells were then taken and immediately centrifuged at 10,000 × g in a Beckman centrifuge for 15 minutes. Next, the supernatant is decanted and a solution (50% EtOH, 300 mM NaCl, 2 mM EDTA) is contacted with the pellet to strip the compaction agents from the DNA now pelleted with the cellular waste. The bottle is vortexed, centrifuged, and the stripping solution is decanted. The pellet is then washed with 70% EtOH, decanted and then resuspended (the now soluble nucleic acids) in 1× TAE (40 mM Tris HCl, 1 mM EDTA, 40 mM NaAc at pH 8.0). The sample is then vortexed, centrifuged, and the supernatant is decanted to a new tube.

This supernatant is enriched in genomic DNA.

### Example 3

### Mechanical Lysis of Bacterial Cells for the Recovery of Bacterial Artificial Chromosomes (BACs)

Using the same procedure detailed in Example 1, a lysate is used to purify BACs from bacterial cells (containing the BAC of interest). Bacterial cell paste is then taken and resuspended in an aqueous compaction protection solution, 10 mL per gram of wet cells, composed of a non-ionic detergent (1% Brij 58), spermidine trihydrochloride (optimally 10 mM), and 20 mM Tris HCl at pH 8.0. This solution is vortexed to resuspend the bacteria in the buffer and then run through a French cell press (SLM Aminco, cat # PC-160) at an average pressure of 11,000 psi. These lysed cells were then taken and immediately centrifuged at 10,000 × g in a Beckman Centrifuge for 15 minutes. Next, the supernatant is decanted and a solution (50% EtOH, 300 mM NaCl, 20 mM EDTA) is contacted with the pellet to strip the compaction agents from the DNA now pelleted with the cellular waste. The bottle is vortexed, centrifuged, and the stripping solution is decanted. The pellet is then washed with 70% EtOH, decanted and then resuspended (the now soluble nucleic acids) in 1× TAE (40 mM Tris HCl, 1 mM EDTA, 40 mM NaAc at pH 8.0). The sample is then vortexed, centrifuged, and the supernatant is decanted to a new tube. This supernatant is enriched in BAC DNA.

### Example 4

### Bacterial Lysis Only

Using a protocol similar to Example 1, a lysate can be created with little or no purification other than lysis of bacterial cells.

Bacterial cell paste is suspended in an aqueous compaction protection solution, 10 mL per gram of wet cells, composed of a non-ionic detergent (1% Brij 58), spermidine trihydrochloride (optimally 10 mM), and 20 mM Tris HCl at pH 8.0. This solution is vortexed to resuspend the bacteria in the buffer and then run through a French cell press (SLM Aminco, cat # PC-160)) at an average pressure of 11,000 psi. To these lysed cells 1 volume of a high salt solution (1 M NaCl, 25 mM EDTA, and 50 mM Tris (˜pH 8.0) is added. Then the solution is vortexed and immediately centrifuged at 10,000 × g in a Beckman Centrifuge for 15 minutes. Next, the supernatant is decanted and precipitated with 7 volumes of ice-cold isopropanol.

### Example 5

### Compaction Protection of Plasmid DNA during Sonication

Different shear induced lysis techniques can be used to separate DNA when compaction protection is utilized. Sonication is a classic lysis technique that has been used extensively in the recovery of proteins from cells. A problem with using sonication with nucleic acids is the induced force and heat can degrade nucleic acids. The inventors have used a sonicator to lyse a compaction protected cell suspension.

First bacterial cells containing the plasmid pCMV sport B gal were grown overnight in a 5 mL LB culture. Next, 2 mL of this solution is centrifuged and the pellet is resuspended in 200 uL of compaction protection solution (composed of a nonionic detergent (1% Brij 58), spermidine trihydrochloride (optimally 10 mM), and 20 mM Tris HCl at pH 8.0). This solution is sonicated on ice using a 1 second cycle time, 50% Duty cycle and an output control setting of 4. Next, the solution is centrifuged at 14,000 × g in an Eppendorf Microfuge (model 5415C) for 5 minutes.

Next, the supernatant is decanted and a solution (50% EtOH, 300 mM NaCl, 20 mM EDTA) is contacted with the pellet to strip the compaction agents from the DNA now pelleted with the cellular waste. The bottle is vortexed, centrifuged, and the stripping solution is decanted. The pellet is then washed with 70% EtOH, decanted and then resuspended (the now soluble nucleic acids) in 1 × TAE (40 mM Tris HCl, 1 mM EDTA, 40mM NaAc at pH 8.0). The sample is then vortexed, centrifuged, and the supernatant is decanted to a new tube and analyzed by gel electrophoresis.

### Example 6

### Mini-prep Using Glass Beads

A separation for nucleic acids has been demonstrated using small glass beads and a vortex to homogenize a compaction-protected bacterial cell suspension. First bacterial cells containing the plasmid pCMV sport B gal were grown overnight in a 5 mL LB culture. Next, 2 mL of this solution is centrifuged in a microfuge tube and the pellet is resuspended in 200 uL of compaction protection/bead solution composed of a non-ionic detergent (1% Brij 58), spermidine trihydrochloride (optimally 20 mM), and 40 mM Tris HCl at pH 8.0 containing 50% v/v acid washed/silanized glass beads (Supelco, 75 ˜m, cat # 59201). This solution is vortexed at max speed on a Fisher tabletop vortexer for 1 minute. Next, the solution is centrifuged at 14,000 × g in an Eppendorf Microfuge (model 5415C) for 5 minutes.

Next, the supernatant is decanted and a solution (50% EtOH, 300 mM NaCl, 20 mM EDTA) is contacted with the pellet to strip the compaction agents from the DNA now pelleted with the cellular waste. The bottle is vortexed, centrifuged, and the stripping solution is decanted. The pellet is then washed with 70% EtOH, decanted and then resuspended (the now soluble nucleic acids) in 1 × TAE (40 mM Tris HCl, 1 10 mM EDTA, 40 mM NaAc at pH 8.0). The sample is then vortexed, centrifuged, and the supernatant is decanted to a new tube.

This example also works well when using filtration in the place of centrifugation; especially since the glass beads work as a filter aid.

### Example 7

### Protection of Large Nucleic Acids during Homogenization

Currently, many mammalian cells, tissues, and plants can be lysed using homogenization. High shear forces can cause physical damage to nucleic acids. Using compaction protection, degradation is minimized.

### Example 8

### 96-well Plasmid/Genomic DNA Purification

Using currently available technology, bacteria is mechanically lysed in a 96 well plate format using shear-induced lysis. A microplate is used with either an extremely fine hole or a very small galling valve in each plate. When a compaction-protected cell suspension is placed into this plate and a force is applied (e.g. by vacuum or centrifugation) the suspension will be driven through the constriction and lysis will occur. In another embodiment, Example 6 can be used in the 96 well plate format.

### Example 9

### Mechanical Lysis of Yeast for the Recovery of Plasmid DNA, Genomic DNA, and/or Yeast Artificial Chromosomes (YACs)

Yeast can be used to produce plasmid DNA, YACs and other nucleic acids. Using the procedures outlined in Example 1, yeast cell mass is processed in the same way as bacterial cell mass for the separation of nucleic acids.

### Example 10

### Secondary Extraction with PEG Precipitation for Plasmid DNA Low in Genomic DNA and Vice Versa

In a similar application to Example 1, plasmid DNA is directly resuspended into a polyethylene glycol (PEG) solution and thus easily adds another level of purification to the process. PEG precipitation of nucleic acids is well known and capable of precipitating chromosomal DNA and some RNA while leaving plasmid DNA in solution. One can resuspend a pellet containing compaction protected DNA in a PEG containing solution to solubilize plasmid DNA from the pellet while leaving genomic DNA and some RNA precipitated. It can be included as a separate process step after most of the previous examples.

### Example 11

### Secondary Extraction with Ammonium Sulfate Precipitation for Plasmid DNA Low in Genomic DNA, RNA, and Protein

Ammonium-sulfate precipitation can be used to precipitate gem DNA and RNA from solution. This works well as a method of removing the genomic DNA and RNA from a mechanically lysed sample.

### Example 12

### Compaction Protection of Nucleic Acids During Heat Lysis of Nucleic Acids

Heat lysis of bacteria for the recovery of plasmid DNA has been used for an extended period. This application deals with protection of nucleic acids during the application of high temperatures. This temperature increase can denature and damage large DNA molecules. Using compaction protection, the amount of damage incurred by DNA during heat lysis is limited.

### Example 13

### Protection of Nucleic Acids from Nucleases, Restriction Enzymes and Other Nucleic Acid Modifying Enzymes Via Compaction Protection

Another use of compaction protection is to protect nucleic acids from nuclease damage. It is found that compaction agents, even if the DNA is not condensed, inhibit the actions of nucleases. Thus, a small amount of compaction agent in a solution limits nuclease activity.

### Example 14

### Use of Compaction Precipitation to Protect mRNA from Degradation During Purification

In general, the most challenging nucleic acid separation is the purification of intact messenger RNA from host cells. This form of nucleic acid is very sensitive to pH and nucleases. Compaction protection aids in the purification of intact messenger RNA.

### Example 15

### Use of Compaction Agents in a Storage Buffer for the Long-Term Protection of Nucleic Acids from Degradation

Long-term protection of nucleic acids is a problem with new vaccines and therapeutics coming onto the market. A storage solution containing a compaction agent reduces damage by shear, nucleases, pH, etc. A small, non-toxic molecule stabilizes the nucleic acid structure allowing for longer storage periods.

### Example 16

### Protection of Nucleic Acids During Sterile Filing

One problem with nucleic acid purification, for pharmaceutical purposes, is degradation during the final filling of product into containers for distribution. Compaction agents protect the nucleic acids being aliquoted into sterile containers from shear-induced degradation.

### Example 17

### Combined Compaction Protection and Compaction-Based Purification

E. coli strain JM109 containing plasmid pCMV sport B gal (Gibco BRL) is grown in a 20 L Applikon fermenter, harvested, pelleted by centrifugation and stored at −80° C. The compaction protection buffer consisting of 0.5% w/v Brij 58 with 20 mM spermidine in 20 mM Tris HCl at pH 8.0 (“compaction protection solution”) is added directly to frozen cell mass at 10 mL per gram of wet cells (resulting optical density ca. 80). The non-ionic detergent Triton X100 (1% w/v) performed equivalently to 0.5% Brij 58.

The mixture is vortexed to resuspend the cells, allowed to stand for five minutes, and mechanically lysed using a French press (PC-160, SLM Aminco) at a pressure of 4,000-11,000 psi (see below). The next step is to add one volume of 1.2 M NaCl in 20 mM Tris HCl, pH 8.0 to the spermidine-protected lysate. The plasmid was then released into the supernatant, the mixture was centrifuged (10,000 × g, 15 minutes) and the supernatant decanted. For electrophoresis, the supernatant was desalted by addition of 0.7 volumes of isopropanol, 1 hour incubation at −20° C., and centrifugation at 10,000 × g for 15 minutes. The pellet was then resuspended in 1× TAE (40 mM Tris HCl, pH 8.0, 40 mM acetic acid, and 1 mM EDTA) and analyzed by electrophoresis on a pre-cast E-gel (Invitrogen).

In a second approach, the lysate is centrifuged at 10,000 × g for 1-5 minutes. The supernatant is discarded and the compaction-precipitated DNA and cell fragments are exposed to stripping solution (50% ethanol, 300 mM NaCl with 10 mM EDTA; 1 mL per 10 -mL initial lysate) to remove the spermidine from the DNA while keeping it insoluble. This solution is then vortexed and centrifuged at 10,000 × g for 5 minutes. The resulting RNA depleted pellet is then washed with 70% ethanol and resuspended in 1 × TAE for further processing, optionally including selective plasmid precipitation using spermidine.

### Example 18

### Protection of Nucleic Acids for Purer Product

The second approach, while more complex, yields a purer product. In this approach, the lysate of Example 17 is centrifuged at 10,000 × g for 15 minutes. The supernatant is discarded and the compaction-precipitated DNA and cell fragments were exposed to stripping solution (50% ethanol, 600 mM NaCl with 10 mM EDTA; 1 mL per 10 mL initial lysate) to remove the spermidine from the DNA while keeping it insoluble. This solution is then vortexed and centrifuged at 10,000 × g for 5 minutes. The resulting RNA-depleted pellet is then washed with 70% ethanol and resuspended in 1 × TAE for further processing, optionally including selective plasmid precipitation using spermidine.

### Example 19

### Protein Isolation from Compaction-Protected Lysate

E. coli JM109 strain containing plasmid DNA encoding the production of recombinant cytochrome b5 is grown in LB medium at 37° C. in a 20 L Applikon fermentor (20 liter in-situ sterilizable bioreactor). Overall fermentation time continues for about 12 hours and the cells grow to an OD600 of about 20. The fermentor is harvested and the cells are pelleted at 4000 rpm in a Beckman centrifuge (6 L capacity rotor) for 30 minutes. Then the resulting pellets are optionally placed into plastic bags and heat-sealed and frozen to make crisps. The yield of the fermentation is approximately 450 grams of wet cell paste. This bacterial paste is then taken and resuspended in an aqueous compaction protection solution, 10 mL per gram of wet cells, composed of a non-ionic detergent ( 1% Brij 58), spermine (10 mM), and 30 mM Tris HCl at pH 8.0.

This solution is vortexed to suspend the bacteria in the buffer and then run through a French cell press (SLM Aminco, cat # PC-160) at an average pressure of 11,000 psi. These lysed cells are centrifuged at 10,000 × g in a Beckman centrifuge for 15 minutes. Next, the supernatant is decanted and passed through a series of chromatography steps to isolate the desired protein Loading flow rate is substantially improved compared to loading of a column with lysate prepared without compaction agent in the lysis buffer.

### Modifications

Specific compositions, methods, or embodiments discussed are intended to be only illustrative of the invention disclosed by this specification. Variations on these compositions, methods, or embodiments are readily apparent to a person of skill in the art based upon the teachings of this specification and are therefore intended to be included as part of the invention disclosed herein. For example, other preferred applications comprise:

A. A method of preparing substantially purified DNA by adding an effective amount of a compaction agent to a cell mass comprising DNA; lyzing the cell mass to release DNA; and separating DNA having a content of RNA of less than 10% by weight of the lysate.

B. A method for the production of purified nucleic acids, from a cell mass comprising nucleic acids, comprising in combination the following steps:

a. adding an amount of a compaction agent, effective to cause compaction of the nucleic acid;

b. lysing the cell mass to liberate nucleic acids;

c. optionally adjusting the ionic strength, pH and/or plasmid concentration

d. optionally precipitating some non DNA moieties.;

e. precipitating a substantial fraction of the nucleic acids away from contaminating protein by addition of an effective amount of a compaction agent.

C. A composition of matter comprising DNA, substantially free of added nucleases, and, containing less than about 3% RNA by weight and at least one part per trillion by weight trace of cationic compaction agent.

D. A method of treatment of a cell mass comprising desired RNA product and contaminating DNA comprising mechanical lysis of the mixture in the presence of a compaction agent to release RNA and precipitate at least a portion of the contaminating DNA.

E. A method for the production of purified nucleic acids, from a cell mass comprising nucleic acids containing DNA, comprising in combination the following steps:

a. adding an amount of a compaction agent. effective to compact the nucleic acid;

b. lysing the cell mass to liberate nucleic acids;

c. optionally adjusting the ionic strength, pH and/or plasmid concentration;

d. optionally precipitating some non DNA moieties;

e. precipitating a substantial fraction of the DNA away from contaminating RNA and protein by addition of an effective amount of a compaction agent.

F. A composition of C above additionally comprising less than 0.0001 weight % RNAse.

G. A composition of claim C above comprising a plasmid DNA encoding proteins for use as a vaccine.

H. A composition of G above wherein the protein comprises influenza protein.

I. A method according to C′ above wherein DNA is separated from endotoxin to a level of less than 0.1 EU/μg plasmid DNA.

K. A method according to B or E above for producing ribosomal RNA, chromosomal DNA, plasmid DNA, aptamers, artificial RNA, or mRNA, or BACs or other natural or synthetic nucleic acids.

L. The method of A above comprising producing plasmid having a content of ribonucleases which is undetectable by standard assays.

M. The composition of C above additionally comprising a content of eukaryotic ribonucleases of less than 0.1% by weight.

N. The method of A above comprising producing plasmid having a content of eukaryotic ribonucleases of less than 0.001% by weight.

O. The method of A above in which the addition of the compaction agent comprises the addition of two or more different mixed compaction agents whereby improved separation efficiency results.

P. The method of B or E above further comprising subsequent chromatographic column purification wherein prior use of compaction agents enhances the overall loading capacities of plasmid DNA on anion-exchange columns by elimination of the majority of contaminating RNA and other biomolecules, which would otherwise impair the subsequent chromatography.

Q. A method according to A above additionally comprising stripping the compaction agent by a stripping method selected from the group comprising high salt addition and/or a pH shift.

R. A composition for the recovery of DNA comprising a mixture of combined reagents, one of which lyses and one of which compacts DNA to clarify a cell mass.

S. A composition according to R above in which the lysing agent comprises a nonionic detergent.

T. A method according to B or E above in which lysing cells is accomplished at a low salt (less than about 300 mM) concentration which is applied to lyse RNA-containing cells.

U. A method according to B or E above wherein the method is applied to remove large nucleic acid molecules from low ionic strength bacterial lysates.

V. A method according to B or E above comprising a lyzing technique selected from the group consisting of use of: mechanical lysing e.g. French cell press, homogenizer, microfluidizer; addition of nonionic detergent, heating, lysozyme addition, freeze-thaw or any other relatively low ionic strength lysis technique to produce nucleic acid free lysates for later protein recovery.

W. A method according to A above comprising simultaneous application of the method in parallel mini-prep procedures for a plurality of cell masses.

X. A method according to B above producing pharmaceutical grade plasmid DNA with an RNAse level, chromosomal DNA level, contaminating protein level, an endotoxin level and a RNA level below the guidelines set forward by the Food and Drug Agency at website: http://www.fda.org.

Y. A method according to B above additionally comprising a further separation step comprising one or more techniques selected from the group consisting of: precipitation and resuspension, filtration and adsorption for production of more pure product.

AA. method according to B above comprising addition of about 0.001 to 20 mM of a compaction agent selected from the group consisting of: basic polypeptides, polyamines, trivalent and tetravalent metal ions, or manganese salts.

BB. The method of B above wherein the cell mass comprises nucleic acid or a synthesized analog.

CC. The method of B above wherein the cell mass comprises Gram-positive bacteria, Gram-negative bacteria, yeast, eukaryotes, synthesized nucleic acids, Archaea, bacteria, protozoa, phages, other viruses, human cells, body fluids, mixtures of cells, tissues, or environmental samples.

EE. A method according to A comprising precipitating a substantial fraction of the DNA away from contaminating RNA and protein by addition of the compaction equivalent of one volume of from 1 to 40 mM spermidine in the form of a compaction agent.

GG. A composition of C above comprising less than about 0.1 Units endotoxin per microgram plasmid DNA (EU/ug or IE/ug).

HH. A method according to B above producing a product comprising less than 0.3 Units endotoxin per microgram plasmid DNA (EU/ug or IE/ug).

II. A composition of C above comprising less than 0.3 Units endotoxin per microgram plasmid DNA (EU/ug or IE/ug).

JJ. A composition of C above comprising less than 0.1 Units endotoxin per microgram plasmid DNA (EU/ug or IE/ug).

KK A kit comprising compaction agent, lysing agent and/or lyzing apparatus and other reagents and apparatus designed for the purification of nucleic acids from cell mass, lysates or synthetic solutions.

LL. A purification kit for plasmid DNA according to KK above-comprised of lysis solutions, a resuspension solution, a compaction agent-based precipitation solution, a stripping solution and optionally a final resuspension solution.

MM. A purification kit for total RNA according to KK above comprised of a lysis solution and/or lyzing apparatus; a 1^{st} compaction precipitation solution (which may be optionally combine with the lysis solution); a 2^{nd} compaction precipitation solution; a stripping solution; and optionally a final resuspension solution.

NN. A purification kit for chromosomal or genomic DNA according to KK above comprised of a lysis solution or solutions, a resuspension solution, a compaction agent-based precipitation solution, a stripping solution, and optionally a final resuspension solution.

OO. A purification kit for large RNA fragments according to KK above comprised of a lysis solution; a 1^{st} compaction precipitation solution (which may be optionally combined with the lysis solution); a 2^{nd} compaction precipitation solution; a stripping solution; and optionally a final resuspension solution.

PP. A purification kit for low molecular weight RNA fragments according to KK above comprised of a lysis solution; a 1^{st} compaction precipitation solution (which may be optionally combine with the lysis solution); a 2^{nd} compaction precipitation solution; a 3^{rd} compaction precipitation solution; a stripping solution; and optionally a final resuspension solution.

QQ. A large-scale plasmid DNA purification kit according to KK above comprised of lysis solutions, a resuspension solution, a compaction agent-based precipitation solution, a stripping solution and optionally a final resuspension solution.

RR. A large-scale filtration-based plasmid DNA purification kit according to QQ above comprised of lysis solutions, a resuspension solution, a compaction agent-based precipitation solution, a stripping solution and optionally a final resuspension solution.

SS. A purification kit for nucleic acids according to KK above comprised of a lysis solution; a 1^{st} compaction precipitation solution (which may be optionally combined with the lysis solution); optionally a 2 ^{nd} compaction precipitation solution and stripping solution; and an adsorbent.

TT. A purification kit for nucleic acids according to KK above comprised of a lysis solution; a 1^{st} compaction precipitation solution (which may be optionally combine with the lysis solution); optionally a 2^{nd} compaction precipitation solution and stripping solution; and an ion exchange adsorbent.

UU. A purification kit for nucleic acids according to KK above comprised of a lysis solution; a 1^{st} compaction precipitation solution (which may be optionally combined with the lysis solution); optionally a 2^{nd} compaction precipitation solution and stripping solution; and a metal-chelate affinity adsorbent.

VV. A robot-compatible parallel purification kit for nucleic acids according to KK above comprised of a lysis solution; a 1^{st} compaction precipitation solution (which may be optionally combined with the lysis solution); optionally a 2^{nd} compaction precipitation solution and stripping solution; and an adsorbent.

WW. A purification kit for nucleic acids according to KK above comprised of a lysis solution; a 1^{st} compaction precipitation solution (which may be optionally combined with the lysis solution); optionally a 2^{nd} compaction precipitation solution and stripping solution; and a filter.

XX. The use of filtration devices to enhance the speed and usability of kits listed in KK-WW above.

YY. A method of A and B above, in which the compaction agenat also acts to promote cell lysis.

Reference to documents made in the specification is intended to result in such patents or literature being expressly incorporated herein by reference.

What is claimed is:

1. A method of treatment of a mixture comprising cells containing RNA and/or DNA comprising lysis of the mixture in the presence of a compaction agent to release as product at least a portion of the RNA and/or DNA substantially without damage.

2. A method according to claim 1 for producing ribosomal RNA, chromosomal DNA, BAC, YAC, plasmid DNA, aptamer, artificial RNA, or mRNA.

3. A method of protein purification comprising in combination:
A. Lysing cells containing nucleic acids in the presence of a compaction agent;
B. Optionally separating into portions the lysate resulting from A by centrifugation or filtration;
C. Subjecting the resulting lysate or portion of the lysate to further steps to isolate a substantially-purified protein product;
Wherein said compaction agent is present in an amount at least effective to render substantially insoluble a portion of said nucleic acid.

4. A composition comprising protein suspended in a fluid comprising a compaction agent.

5. A method of treatment of a mixture comprising desired RNA product and contaminating DNA comprising lysis of the mixture in the presence of a compaction agent selected from the group consisting of: basic polypeptides, polyamines, trivalent and tetravalent metal ions, to precipitate at least a portion of the DNA.

6. A method according to claim 4 for producing ribosomal RNA, chromosomal DNA, plasmid DNA, BAC, YAC, aptamer, artificial RNA, or mRNA

7. The method of claim 1 comprising producing plasmid having an undetectable content of ribonucleases by standard assays.

8. The method of claim 1 comprising producing plasmid having a content of eukaryotic ribonucleases of less than 0.001% by weight.

9. The method of claim 1 in which the addition of the compaction agent comprises the addition of two or more different mixed compaction agents whereby improved separation efficiency results.

10. The method of claim 1 further comprising subsequent chromatographic column purification wherein prior use of compaction agents enhances the overall loading capacities of plasmid DNA on anion-exchange columns by elimination of the majority of contaminating RNA and other biomolecules, which would otherwise impair the subsequent chromatography.

11. A method according to claim 1 additionally comprising stripping the compaction agent by a stripping method selected from the group comprising high alt addition and/or a pH shift.

12. A method according to claim 1 wherein the method is applied to remove large nucleic acid molecules from low ionic strength bacterial lysates.

13. A method according to claim 1 additionally comprising a technique selected from the group consisting of: use of French cell press, addition of nonionic detergent, lysozyme addition, microfluidizer, freeze-thaw or any other relatively low ionic strength lysis technique to produce nucleic acid free lysates for later protein recovery.

14. A method according to claim 1 comprising simultaneous application of the method in parallel mini-prep procedures for a plurality of cell masses

15. A method according to claim 1 additionally comprising a further separation step comprising one or more techniques selected from the group consisting of: precipitation and resuspension, filtration and adsorption for production of plasmid DNA with an RNAse level, chromosomal DNA level, contaminating protein level, an endotoxin level and a RNA level below the guidelines set forward by the Food and Drug Agency.

16. A method according to claim 1 comprising addition of about 0.001 to 50 mM of a compaction agent selected from the group consisting of: basic polypeptides (e.g. polylysine), polyamines (e.g. protamine, spermidine, spermine, cadaverine, etc.), trivalent and tetravalent metal ions (e.g. hexammine cobalt, chloropentammine cobalt, chromium (III)), netropsin, distamycin, lexitropins, DAPI (4′,6 diamino 2-phenylindol), berenil, pentamidine, or manganese chloride.

17. The method of claim 1 wherein the cell mass comprises nucleic acid or a synthesized analog.

18. The method of claim 1 wherein the source of the lysate is selected from the group consisting of Gram-positive bacteria, Gram-negative bacteria, yeast, eukaryote, synthesized nucleic acids, Archaea, bacteria, protozoa, phages, other viruses, human cells, body fluids, mixtures of cells, tissues, or environmental samples.

19.A method according to claim 1 comprising precipitating a substantial fraction of tie DNA away from contaminating RNA and protein by addition of the compaction equivalent of one volume of from 1 to 25 mM spermidine in the form of a compaction agent.

20. A method according to claim 1 additionally comprising stripping the compaction agent by a stripping method selected from the group comprising high salt addition and/or a pH shift followed by additional purification steps.

21. A method according to claim 1 additionally comprising stripping the compaction agent by a stripping method selected from the group comprising high salt addition and/or a pH shift, followed by selective precipitation using compaction agent.
All inventions substantially as described herein.
TABLE A

| | | | |
|---|---|---|---|
| Parameter | Units | Preferred | Most Pref. |
| | | |
|---|---|---|
| Cell Mass | Archaea eukaryotes | Gram-neg |
| | bacterial, Gram-negative | |
| | Gram-positive | |
| | phage, yeast | |
| Product: | DNA, RNA, Assay | plasmid |
| | NA-binding protein | DNA |
| | enzymes, cosmids, | |
| | YACs, BACs | |
| | Plasimd | |

Compaction Agent: Preferred; basic polypeptides (e.g. polylysine), polyamines (e.g. protamine, spermidine, spermine, putrescine, cadaverine, etc.), trivalent and tetravalent metal ions (e.g. hexammine cobalt, chloropentammine cobalt, chromium (III)), netropsin, distamycin, lexitropans, DAPI (4′,6 diamino 2-phenylindol), berenil, pentamidine, manganese chloride. Most preferred: hexammine cobalt, spermine and spermidine

| | | |
|---|---|---|
| CA Conc. mM | 0.02-2.00 | 0.05-20 |

Lysing Agent: detergent, nonionic detergent, heat, French press, sonicator, homogenizer, microfluidizer, freeze/thaw, toluene, organic solvent, amines, quaternary amines, enzyme, lysozyme, lysostaphin, osmotic shock, chloroform, extruder, bead mill, microneedles, acid, alkali, phage protein
Preferred: French press, homogenizer, bead mill, microfluidizer, freeze/thaw, enzyme, heat
Most preferred: French press, homogenizer, microfluidizer, heat

| ““Conc.:wt % | | 0.5-2 | .05-.5 |
|---|---|---|---|
| pH | varies | 6-8 | 7 |
| Ionic Strength: mM | | 0-200 | 0-50 |
| (Before Compaction) | | | |
